SEQUENCE LISTING (13 sequences)

SEQ ID NO: 1 - DNA, 22 bp, artificial sequence
  primer HRH4-F
ctccatattctctgttcacaat

SEQ ID NO: 2 - DNA, 22 bp, artificial sequence
  primer HRH4-R
tggatacaaaagaggattgaca

SEQ ID NO: 3 - DNA, 24 bp, artificial sequence
  H4-Probe
aacaggtcctaaatcagtttggta

SEQ ID NO: 4 - DNA, 21 bp, artificial sequence
  primer GAPDH-F
gacaacagcctcaagatcatc

SEQ ID NO: 5 - DNA, 18 bp, artificial sequence
  primer GAPDH-R
cgccacagtttcccggag

SEQ ID NO: 6 - DNA, 24 bp, artificial sequence
  G-Probe
actcatgaccacagtccatgccat

SEQ ID NO: 7 - DNA, 21 bp, artificial sequence
  primer HRH4-F
gccagatactaatagcacaat

SEQ ID NO: 8 - DNA, 22 bp, artificial sequence
  primer HRH4-R
ccacaaaagctaaaatgaccaa

SEQ ID NO: 9 - DNA, 22 bp, artificial sequence
  H4-Probe
aagcactcgtgttactttagca

SEQ ID NO: 10 - DNA, 88 bp, Artificial Sequence
  pGM-T fragment
tgtaatacgactcactatagggcgaattgggcccgacgtcgcatcctcccggccgccatggcggccgcgggaattcgtatccaagatt

SEQ ID NO: 11 - DNA, 87 bp, Artificial Sequence
  pGM-T fragment
atcttggatacgaattcccgcggccgccatggcggccgggagcatgcgacgtcgggcccaattcgccctatagtgagtcgtattaca

SEQ ID NO: 12 - DNA, 106 bp, Artificial Sequence
  pGM-T fragment
atcttggatacactagtgaattcgcggccgcctgcaggtcgaccatatgggagagctcccaacgcgttggatgcatagcttgagtattctatagtgtcacctaaat

SEQ ID NO: 13 - DNA, 107 bp, Artificial Sequence
  pGM-T fragment
atttaggtgacactatagaatactcaagctatgcatccaacgcgttgggagctctcccatatggtcgacctgcaggcggccgcgaattcactagtgtatccaagatt

(19) United States
(12) Patent Application Publication
(10) Pub. No.: US 2024/0150832 A1
(43) Pub. Date: May 9, 2024

(54) REAGENT FOR DETECTING EXPRESSION LEVEL OF HUMAN HISTAMINE RECEPTOR HRH4 MRNA, KIT AND DETECTION METHOD

(71) Applicant: HANGZHOU ZHEDA DIXUN BIOLOGICAL GENE ENGINEERING CO., LTD, Zhejiang (CN)

(72) Inventors: Shandong WU, Zhejiang (CN); Yi LIU, Zhejiang (CN); Zhoujie WU, Zhejiang (CN); Xuehan JIANG, Zhejiang (CN); Xukai YANG, Zhejiang (CN); Meijie WANG, Zhejiang (CN); Weiyue CAI, Zhejiang (CN)

(21) Appl. No.: 18/282,159

(22) PCT Filed: Aug. 4, 2021
(86) PCT No.: PCT/CN2021/110526

§ 371 Date: Sep. 14, 2023

(51) Int. Cl. C12Q 1/6876

(52) U.S. Cl. CPC C12Q 1/6876; C12Q 1/686; C12Q 2600/158

(57) ABSTRACT

The present disclosure provides a reagent for detecting an expression level of a human histamine receptor HRH4 mRNA, a kit and a detection method. In the present disclosure, the reagent includes a specific primer and a probe for a human histamine receptor HRH4, the specific primer includes an HRH4-F and an HRH4-R, and the probe includes an H4-Probe; and the HRH4-F has a nucleotide sequence shown in SEQ ID NO. 1, the HRH4-R has a nucleotide sequence shown in SEQ ID NO. 2 and the H4-Probe has a nucleotide sequence shown in SEQ ID NO. 3. In the present disclosure, a kit for one-step detection and a detection method based on the reagent are prepared, and the expression level of the HRH4 mRNA can be one-step quantitatively detected with simple operation and short detection time.

### CROSS-REFERENCE TO RELATED APPLICATION

This application is a national stage application of International Patent Application No. PCT/CN2021/110526, filed on Aug. 4, 2021, which is incorporated by reference herein in its entirety.

### REFERENCE TO SEQUENCE LISTING

A computer readable TXT file entitled “SEQUENCE LISTING”, that was created on Aug. 28, 2023, with a file size of about 3022 bytes, contains the sequence listing for this application, has been filed with this application, and is hereby incorporated by reference in its entirety.

### TECHNICAL FIELD

The present disclosure belongs to the technical field of biological detection, and specifically relates to a reagent for detecting an expression level of a human histamine receptor HRH4 mRNA, a kit and a detection method.

### BACKGROUND

Histamine is an active amine compound widely present in animals and plants, is formed by the decarboxylation of a histidine, and is usually stored in the tissues. The histamine is an important chemical conductive substance in the body, can affect many cell reactions, including allergies, inflammatory reactions, and gastric acid secretion, etc. When the body is stimulated to trigger an antigen-antibody reaction, the cell membrane permeability of mast cells changes to release histamine, and the histamine interacts with a histamine receptor to produce pathophysiological effects. The synthesis of a histamine mainly occurs in mast cells, basophils, lungs, skin and gastrointestinal mucosa, and is consistent with the tissues that store the histamine. The histamine, like other transmitters, binds to specific receptors on target cells, thereby changing the biological activity of cells and exerting wide physiological or pathological effects. The inflammatory effect of histamine, namely the effect of histamine on the immune homeostasis in the body depends on the expression and activity of currently-known 4 histamine receptors. The 4 histamine receptors are named in the order of discovery: a histamine 1 (H1) receptor, a histamine 2 (H2) receptor, a histamine 3 (H3) receptor, and a histamine 4 (H4) receptor; where, the H4 receptor is predominantly distributed in the immune system or hematopoietic-related tissues or cells, and is highly expressed in bone marrow, peripheral hematopoietic cells and other sites related to inflammation. Therefore, the H4 receptor has become a new therapeutic target in immune diseases such as allergic reactions and asthma. The H4 receptor is involved in the chemotaxis of mast cells, eosinophils, and dendritic cells, as well as the production of cytokines in T cells and dendritic cells, and is involved in inflammatory reactions.

Most patients with allergic diseases use symptomatic treatment based on the experience of doctors and use antihistamines to relieve allergic symptoms. The antihistamines that target the H1 receptor are effective for some allergic patients, but still have limitations. H4 receptor antagonists can have a desirable therapeutic effect on patients that have ineffective treatment effect with H1 receptor antagonists. However, the treatment with antihistamines is not effective for all patients, and cannot quantify the therapeutic effect; and long-term medication will also produce a series of side effects. There are no relevant studies and records on this at present.

### SUMMARY

In view of this, the present disclosure provides a reagent for detecting an expression level of a human histamine receptor HRH4 mRNA, a kit and a detection method. The expression level of the HRH4 mRNA can be detected using an RNA one-step method. The present disclosure provides a detection method with high accuracy, wide detection range and high sensitivity for the detection of HRH4 proteins.

In order to realize the above objective, the present disclosure provides the following technical solutions:
The present disclosure provides a reagent for detecting an expression level of a human histamine receptor HRH4 mRNA, including a specific primer and a probe for a human histamine receptor HRH4, where the specific primer includes an HRH4-F and an HRH4-R, and the probe includes an H4-Probe; and
the HRH4-F has a nucleotide sequence shown in SEQ ID NO. 1, the HRH4-R has a nucleotide sequence shown in SEQ ID NO. 2 and the H4-Probe has a nucleotide sequence shown in SEQ ID NO. 3.

Preferably, the reagent may further include a specific primer and a probe of a reference gene GAPDH, where the specific primer of the GAPDH may include a GAPDH-F and a GAPDH-R, and the probe of the GAPDH may include a G-Probe; and

the GAPDH-F may have a nucleotide sequence shown in SEQ ID NO. 4, the GAPDH-R may have a nucleotide sequence shown in SEQ ID NO. 5 and the G-Probe may have a nucleotide sequence shown in SEQ ID NO. ID NO. 6.

Preferably, 5′-ends of the H4-Probe and G-Probe may be separately labeled with different fluorescent reporter groups, and 3′-ends of the H4-Probe and G-Probe may be labeled with a same quenching group or different quenching groups.

Preferably, the fluorescent reporter group may include a 6-carboxyfluorescein (FAM) and a 2,7-dimethyl-4,5-dichloro-6-carboxyfluorescein (JOE), and the quenching group may include a Black Hole Quencher-1 (BHQ1).

Preferably, the HRH4-F, the HRH4-R, the H4-Probe, the GAPDH-F, the GAPDH-R and the G-Probe in the reagent may have a concentration of 2.25 nM, 1.5 nM, 1.5 nM, 1 nM, 1 nM and 1.5 nM, respectively.

The present disclosure further provides a kit for one-step detection of an expression level of a human histamine receptor HRH4 mRNA, including the reagent, a PCR reaction solution, an enzyme mixed solution, a carboxy-X-rhodamine (ROX) reference dye and nuclease-free water.

Preferably, the PCR reaction solution may include a deoxy-ribonucleoside triphosphate (dNTP) mix, MgCl₂ and a buffer; and

the enzyme mixed solution may include a Thermus aquaticus (Taq) enzyme, a reverse transcriptase, a ribonuclease (RNase) inhibitor and a Taq enzyme antibody with a mass ratio of 15:5:4:1.

Preferably, the kit may further include an RNA standard of the human histamine receptor HRH4.

The present disclosure further provides a method for detecting an expression level of a human histamine receptor HRH4 mRNA in one step based on the reagent or the kit, including the following steps: preparing a reaction system using an RNA standard of the human histamine receptor HRH4 as a template, conducting a quantitative real-time polymerase chain reaction (qRT-PCR), and constructing a standard curve using a logarithmic value of a copy number as an abscissa and using a Ct value as an ordinate;

preparing a same reaction system using an RNA extracted from a sample as a template, conducting a same qRT-PCR, and determining the expression level of the human histamine receptor HRH4 mRNA using the standard curve.

Preferably, the reaction system, calculated in 20 μL, may include: 2.4 μL of the nuclease-free water, 10 μL of the PCR reaction solution, 0.5 μL of the enzyme mixed solution, 0.5 μL of the ROX reference dye, 2 μL of the reagent and 5 μL of the template; and

Preferably, a qRT-PCR program may include: 42° C. for 30 min; 95° C. for 1 min; 95° C. for 5 s, and 60° C. for 31 s, 40 cycles.

Preferably, the standard curve is y=−3.177x+34.178, R²=0.996.

The present disclosure further provides use of the reagent or the kit as a basis in preparing a drug for treating an immune disease or as a tool in dynamically monitoring a therapeutic effect.

Compared with the prior art, the present disclosure has the beneficial effects as follows: the present disclosure provides a reagent for detecting an expression level of a human histamine receptor HRH4 mRNA, and a kit for one-step detection of the expression level of the HRH4 mRNA is prepared based on the reagent. The expression level of the HRH4 mRNA in human blood, nasal secretions, bronchial irrigating fluid, saliva, and tear samples can be one-step quantitatively detected using the reagent or the kit with simple operation and short detection time. The present disclosure provides a kit product that can guide the medication and accurately quantify the efficacy for H4 antihistamines.

In the present disclosure, the one-step detection is conducted based on the reagent and the kit without separate reverse transcription, which greatly reduces the risk of causing aerosol pollution. Compared with immunological detection methods, the detection method provided by the present disclosure has high sensitivity, can detect low-concentration clinical samples, can sensitively detect changes in HRH4 content, and has a detection range spanning at least 6 orders of magnitude. Accordingly, the accuracy of the detection results is increased, and at least 80 people can be detected within 1 hour, such that the treatment effect can be dynamically monitored and evaluated in an earlier, more accurate, and faster manner.

### BRIEF DESCRIPTION OF THE DRAWINGS

FIG. 1 is a standard curve of TaqMan real-time fluorescence quantitative RT-PCR for HRH4 mRNA.

FIG. 2 is a result of precision detection, where 1: 1.0×10⁷ copies/μL, and 2: 1.0×10⁴ copies/μL.

FIG. 3 is a result of accuracy detection.

FIG. 4 is a result of sensitivity detection.

FIG. 5 is a result of clinical sample detection, where 1: Case 3 GAPDH mRNA before treatment; 2: Case 3 GAPDH mRNA after treatment; 3: Case 3 HRH4 mRNA before treatment; 4: Case 3 HRH4 mRNA after treatment.

FIG. 6 is a low-precision amplification curve in the case of non-optimal primer and probe designs.

FIGS. 7A and 7B are effect of the enzyme mixed solution on amplification.

FIG. 8 is a plasmid structure map of pGM-T vector.

### DETAILED DESCRIPTION OF THE INVENTION

The present disclosure will be further described below in conjunction with the examples and accompanying drawings.

The present disclosure provides a reagent for detecting an expression level of a human histamine receptor HRH4 mRNA, including a specific primer and a probe for a human histamine receptor HRH4, where the specific primer includes an HRH4-F and an HRH4-R, and the probe includes an H4-Probe; and

the HRH4-F has a nucleotide sequence shown in SEQ ID NO. 1, the HRH4-R has a nucleotide sequence shown in SEQ ID NO. 2 and the H4-Probe has a nucleotide sequence shown in SEQ ID NO. 3.

In the present disclosure, the reagent preferably further includes a specific primer and a probe of a reference gene GAPDH; the specific primer of the reference gene GAPDH preferably includes a GAPDH-F and a GAPDH-R, and the probe of the reference gene GAPDH preferably includes a G-Probe; the GAPDH-F preferably has a nucleotide sequence preferably shown in SEQ ID NO. 4, the GAPDH-R preferably has a nucleotide sequence preferably shown in SEQ ID NO. 5 and the G-Probe preferably has a nucleotide sequence preferably shown in SEQ ID NO. 6. 5′-ends of the H4-Probe and G-Probe are separately labeled with different fluorescent reporter groups, and 3′-ends of the H4-Probe and G-Probe are labeled with a same quenching group or different quenching groups. In an example, the fluorescent reporter group preferably includes an FAM and a JOE, and the quenching group includes a BHQ1, and Shanghai Sunny Biotechnology Co., Ltd. is entrusted to synthesize the specific primer and the probe (Table 1).

TABLE 1
TaqMan real-time fluorescence quantitative
PCR of primer probe

| | Primer sequence | Amplified |
|---|---|---|
| Name | (5′-3′) | fragment |
| HRH4-F | CTCCATATTCTCTGTTCACAAT | 128 bp |
| | (SEQ ID NO: 1) | |
| HRH4-R | TGGATACAAAAGAGGATTGACA | |
| | (SEQ ID NO: 2) | |
| H4-Probe | (FAM)-AACAGGTCCTAAATC | |
| | AGTTTGGTA-(BHQ1) | |
| | (SEQ ID NO: 3) | |
| GAPDH-F | GACAACAGCCTCAAGATCATC | 70 bp |
| | (SEQ ID NO: 4) | |
| GAPDH-R | CGCCACAGTTTCCCGGAG | |
| | (SEQ ID NO: 5) | |
| G-Probe | (JOE)-ACTCATGACCACAGT | |
| | CCATGCCAT-(BHQ1) | |
| | (SEQ ID NO: 6) | |

In the present disclosure, the HRH4-F, the HRH4-R, the H4-Probe, the GAPDH-F, the GAPDH-R and the G-Probe in the reagent have a concentration or number of moles of 2.25 nM, 1.5 nM, 1.5 nM, 1 nM, 1 nM and 1.5 nM, or respectively.

The present disclosure further provides a kit for one-step detection of an expression level of a human histamine receptor HRH4 mRNA, including the reagent, a PCR reaction solution, an enzyme mixed solution, an ROX reference dye and nuclease-free water.

In the present disclosure, the PCR reaction solution preferably includes a dNTP mix, MgCl₂ and a buffer; the dNTP mix is preferably a mixture of a dATP, a dTTP, a dCTP, and a dGTP, which is purchased from Thermo Fisher Scientific (product number: R0192), and has a working concentration of preferably 0.1-1 mM; the MgCl₂ has a concentration of preferably 5-20 mM; and the buffer is a 10-50 mM Tris-HCl buffer (at pH 8.0).

The enzyme mixed solution includes a Taq enzyme, a reverse transcriptase, an RNase inhibitor and a Taq enzyme antibody with a mass ratio of 15:5:4:1 to obtain the best amplification effect.

In the present disclosure, the kit preferably further includes an RNA standard of the human histamine receptor HRH4 to prepare a standard curve.

In the present disclosure, the TaqMan real-time fluorescent quantitative PCR is directly conducted using the RNA as a template without a separate reverse transcription when detecting by the kit; the Taq enzyme is a heat-resistant Taq DNA polymerase, deoxynucleotides in the dNTP are added to a 3-OH terminus one by one using the 3′→5′ polymerase activity of the Taq enzyme and using DNA as a template. Meanwhile, mismatched primer ends can be identified and eliminated using the 5′→3′ exonuclease activity of the Taq enzyme, which is related to the correction function during the replication, nucleotides can also be hydrolyzed from the 5′-end and mismatched nucleotides can also be excised through several nucleotides. In this way, the chain replacement is realized during the chain extension, and the replaced probe is cut off. The reverse transcriptase can reverse transcribe an mRNA into a cDNA for PCR reaction. The RNase inhibitor is used to suppress the activity of an exogenous RNase. The Taq enzyme antibody is an anti-Taq antibody for hot-start PCR, inhibits DNA polymerase activity after binding to the Taq enzyme, and can effectively suppress the non-specific annealing of primers and the non-specific amplification caused by primer dimers under low temperature. The Taq enzyme antibody is denatured during the initial DNA denaturation of the PCR reaction, and the Taq enzyme recovers the activity to realize PCR amplification.

The present disclosure further provides a method for detecting an expression level of a human histamine receptor HRH4 mRNA in one step based on the reagent or the kit, including the following steps: preparing a reaction system using an RNA standard of the human histamine receptor HRH4 as a template, conducting a qRT-PCR, and constructing a standard curve using a logarithmic value of a copy number as an abscissa and using a Ct value as an ordinate;
preparing a same reaction system using an RNA extracted from a sample as a template, conducting a same qRT-PCR, and determining the expression level of the human histamine receptor HRH4 mRNA using the standard curve.

In the present disclosure, preferably a reaction system is prepared using the reagent or the kit to conduct a qRT-PCR; the reaction system, calculated in 20 μL, preferably includes: 2.4 μL of the nuclease-free water, 10 μL of the PCR reaction solution, 0.5 μL of the enzyme mixed solution, 0.5 μL of the ROX reference dye, 2 μL of the reagent and 5 μL of the standard or the RNA; a qRT-PCR program includes: 42° C. for 30 min; 95° C. for 1 min; 95° C. for 5 s, and 60° C. for 31 s, 40 cycles; and an expression of HRH4 mRNA is calculated according to the standard curve prepared with the standard. The standard curve uses a copy number logarithm as an abscissa (x) and a Ct value as an ordinate (y): y=−3.177x+34.178 (R²=0.996). There is no special limitation on the source of the RNA, and the RNA can be extracted from human blood, nasal secretions, bronchial irrigating fluid, saliva or tear samples.

The present disclosure further provides use of the reagent or the kit as a basis in preparing a drug for treating an immune disease or as a tool in dynamically monitoring a therapeutic effect.

In the present disclosure, the immune diseases include preferably allergic diseases. The reagent or the kit can be used to detect the expression level of the histamine H4 receptor (HRH4) mRNA. On one hand, the reagent or kit can determine whether the patient's allergic symptoms are caused by histamine activation of the H4 receptor pathway (since allergic symptoms caused by non-histamine pathways do not express histamine receptors) and guide the dosage (since higher H4 receptor expression level requires higher dosage of H4 receptor antagonist). On the other hand, the effect of H4 receptor antagonist therapy can be dynamically monitored. The use of the present disclosure is preferably the same as that described above, and will not be repeated here.

The reagent for detecting an expression level of a human histamine receptor HRH4 mRNA, the kit and the detection method provided by the present disclosure are described in detail below with reference to the examples, but these examples should not be understood as limiting the claimed protection scope of the present disclosure.

Unless otherwise specified, the test materials involved in the present disclosure are all commercially-available conventional in the art:
a whole-blood total RNA kit (Hangzhou Simgen Biological Reagent Development Co., Ltd., product number: 5201050);
a HiScribe T7 High Yield RNA Synthesis Kit (New England Biolabs, product number: E2050S);
an Applied Biosystems™ 7300 fluorescence quantitative PCR instrument (Thermo Fisher Scientific, USA);
a −80° C. low-temperature refrigerator (Thermo Fisher Scientific, USA);
a high-speed and low-temperature table centrifuge (Eppendorf, Germany); and
a Qubit 3 fluorometer (Thermo Fisher Scientific, USA).

### Example 1

1. Shanghai Sunny Biotechnology Co., Ltd. was entrusted to synthesize the primers and probes shown in Table 1.

2. Preparation of a standard

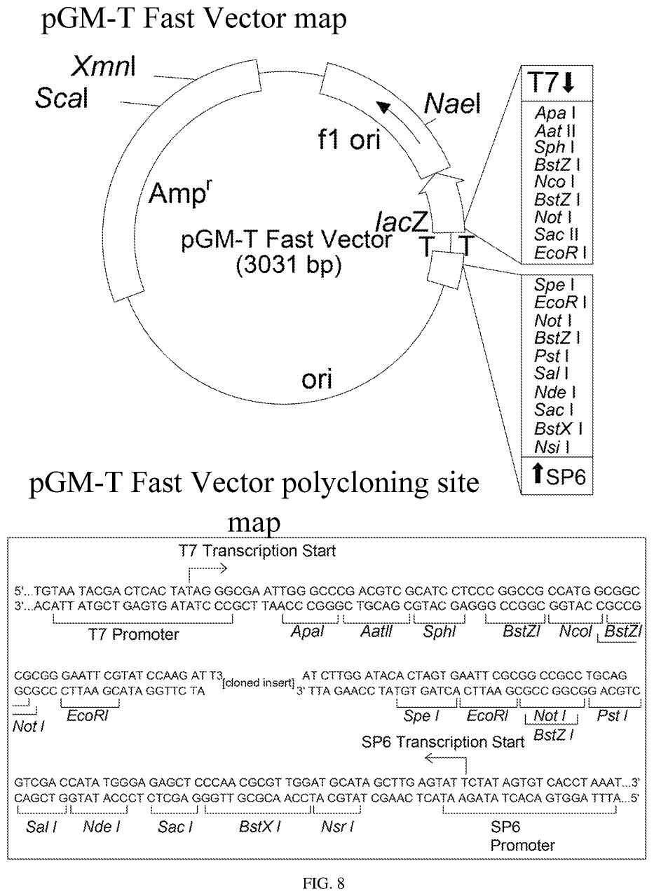

In-vitro transcription: a pGM-T ligation kit [TIANGEN Biotech (Beijing) Co., Ltd., product number: VT202-01] was used, a HRH4 plasmid DNA (constructed and synthesized by entrusting Nanjing GenScript Biotech Co., Ltd., FIG. 8) was constructed using a pGM-T as a vector, and the HRH4 plasmid DNA was transcribed into an mRNA in vitro using a HiScribe T7 High Yield RNA Synthesis Kit (NEW ENGLAND BioLabs, product number: E2040S).

An initial copy number of the RNA was calculated according to a copy number calculation formula: copy number=[6.02×10²³×RNA concentration (ng/μL)×10⁻⁹]/[RNA length (bp)×340]. The HRH4 mRNA was diluted with nuclease-free water to 1.0×10¹⁰ copies/μL to obtain a HRH4 mRNA standard.

3. Extraction and dilution of whole-blood RNA

Whole-blood total RNA was extracted from ethylenediaminetetraacetic acid (EDTA) anticoagulated whole-blood samples with the whole-blood total RNA kit, quantificated with the Qubit 3 fluorometer, and diluted with the nuclease-free water to 20 ng/μL.

4. TaqMan real-time fluorescent quantitative PCR

A 20 μL system was prepared using the standard/whole-blood RNA as a template with: 2.4 μL of the nuclease-free water, 10 μL of the PCR reaction solution, 0.5 μL of the enzyme mixed solution, 0.5 μL of the ROX reference dye, 2 μL of a mixed solution of the primer and the probe, and 5 μL of the standard or the RNA.

A qRT-PCR program was as follow: 42° C. for 30 min; 95° C. for 1 min; 95° C. for 5 s, and 60° C. for 31 s, 40 cycles.

A detection fluorescein was set up: FAM, JOE; a reference fluorescence was: ROX; the reaction system was: 20 μL; and a fluorescence signal collection was: 60° C. for 31 sec.

5. Generation of a standard curve

The HRH4 standard was diluted in a 10-fold gradient using 1.0×10⁸-1.0×10³ copies/ill as a template, 3 replicates were conducted for each dilution, and TaqMan real-time fluorescence quantitative RT-PCR detection was conducted to generate the standard curve.

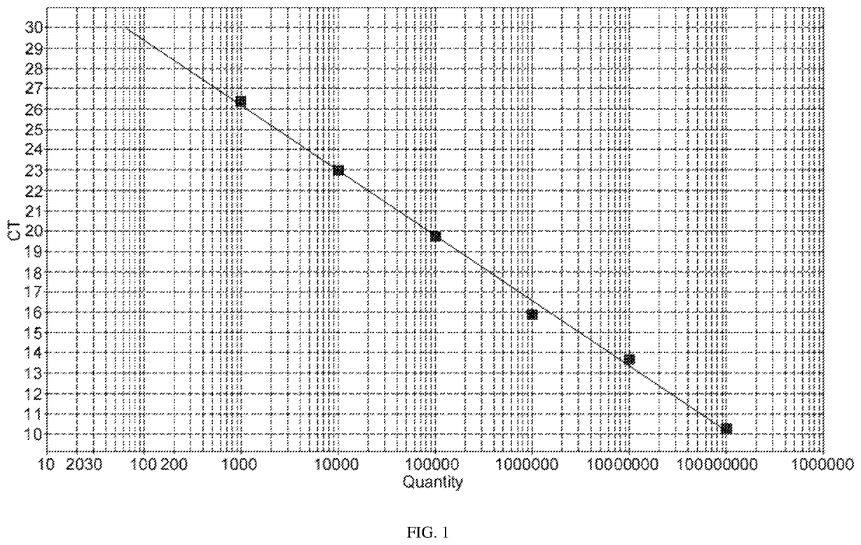

The result is shown in FIG. 1. A copy number logarithm is taken as an abscissa and a Ct value is taken as an ordinate, and a regression equation is obtained: y=−3.177x+34.178 (R²=0.996), indicating the copy number logarithm of a standard equation has very high correlation with the Ct value.

6. Precision detection

1.0×10⁷ copies/μL, and 1.0×10⁴ copies/μL, of standards were taken as templates, 10 replicates were conducted for each concentration; 10 times of TaqMan real-time fluorescent quantitative RT-PCR detections were conducted, the coefficient of variation of the logarithm of each concentration was calculated, respectively; and statistical analysis was conducted to analyze the precision of the detection method.

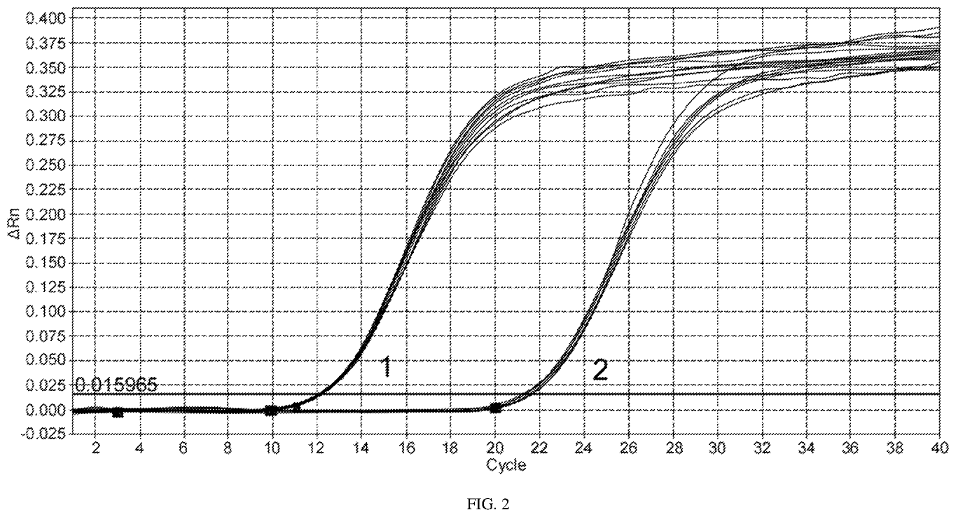

The results are shown in Table 2 and FIG. 2. The coefficient of variation of the logarithm of each concentration is 0.443% and 0.430% separately, which are less than 5%, indicating that the TaqMan real-time fluorescent quantitative RT-PCR detection method established by the present disclosure has excellent precision.

TABLE 2
Precision detection result

| Theoretical | Mean logarithm | | |
|---|---|---|---|
| copy number | of copy number | SD | C.V |
| 1.0 × 10⁷ | 6.907 | 0.031 | 0.443% |
| 1.0 × 10⁴ | 3.988 | 0.017 | 0.430% |

7. Accuracy detection

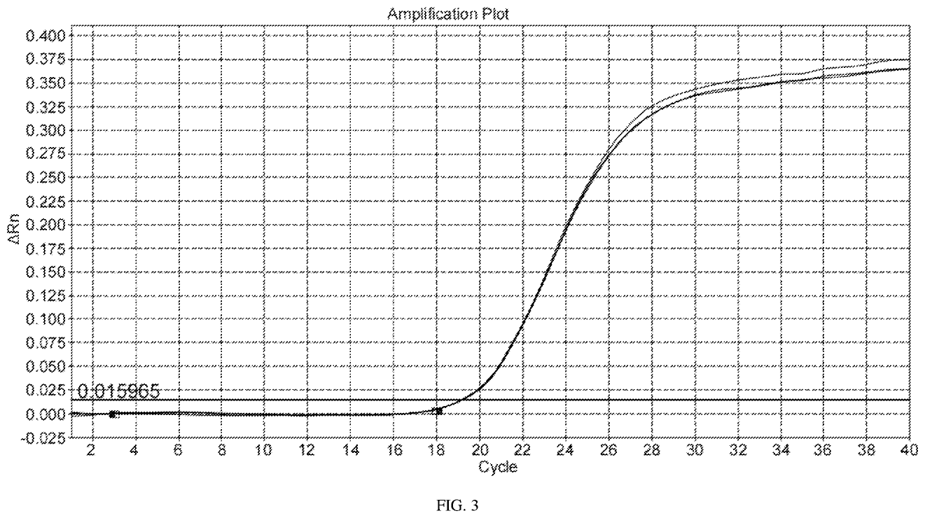

A 1.0×10⁶ copies/μL, of standard was subjected to 30-fold dilution (2 μL 1.0×10⁶ copies/μL, of a standard+58 μL of nuclease-free water) as a template, for 3 replicates; 3 times of TaqMan real-time fluorescence quantitative RT-PCR detections were conducted, and the absolute deviation of the logarithm of each concentration was calculated. The results are shown in FIG. 3. The absolute deviation of the logarithm of each concentration is 0.126, 0.137, and 0.131, respectively, within the range of ±0.5, indicating that the TaqMan real-time fluorescent quantitative RT-PCR detection method established by the present disclosure has excellent accuracy.

TABLE 3
Accuracy detection data

| | | | Theoretical | Theoretical | Abso- |
|---|---|---|---|---|---|
| | | Copy | copy | copy | lute |
| | Results | number | number | number | devia- |
| C_{T} | (copies/μL) | logarithm | (copies/μL) | logarithm | tion |
| 19.409 | 4.454 × 10⁴ | 4.649 | 3.333 × 10⁴ | 4.523 | 0.126 |
| 19.373 | 4.570 × 10⁴ | 4.660 | | | 0.137 |
| 19.393 | 4.505 × 10⁴ | 4.654 | | | 0.131 |

8. Sensitivity detection

A 10.0 copies/μL of standard was taken as a template, for 25 replicates, 25 times of TaqMan real-time fluorescence quantitative RT-PCR detection were conducted to check whether there were amplifications, and the sensitivity of the detection method was analyzed.

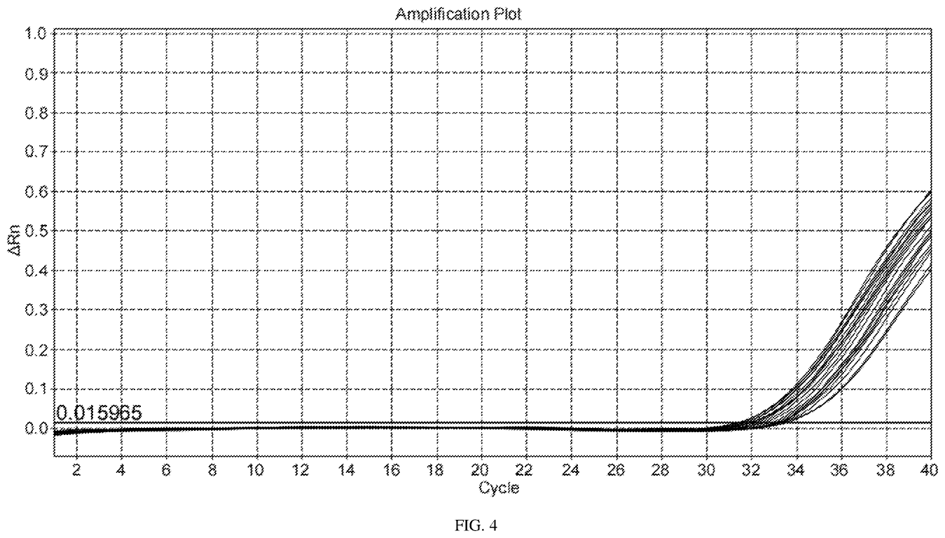

The results are shown in Table 4 and FIG. 4. A total of 25 detection results are obtained, reaching 100%. This indicates that the TaqMan real-time fluorescent quantitative RT-PCR detection method established by the present disclosure has very high sensitivity, and the minimum number of detected copies is less than 10 copies/μL.

TABLE 4
Ct value results of sensitivity detection

| | | | | |
|---|---|---|---|---|
| 32.533 | 32.716 | 33.514 | 32.321 | 31.944 |
| 32.110 | 31.967 | 33.336 | 33.215 | 31.506 |
| 31.673 | 33.513 | 32.747 | 31.747 | 31.961 |
| 31.605 | 32.968 | 33.157 | 31.965 | 32.189 |
| 32.769 | 32.178 | 32.692 | 32.465 | 31.669 |

9. Clinical sample detection

Whole-blood samples of patients and normal persons were taken to extract and dilute whole-blood RNA according to the above steps, and TaqMan real-time fluorescent quantitative RT-PCR detection was conducted according to the above steps.

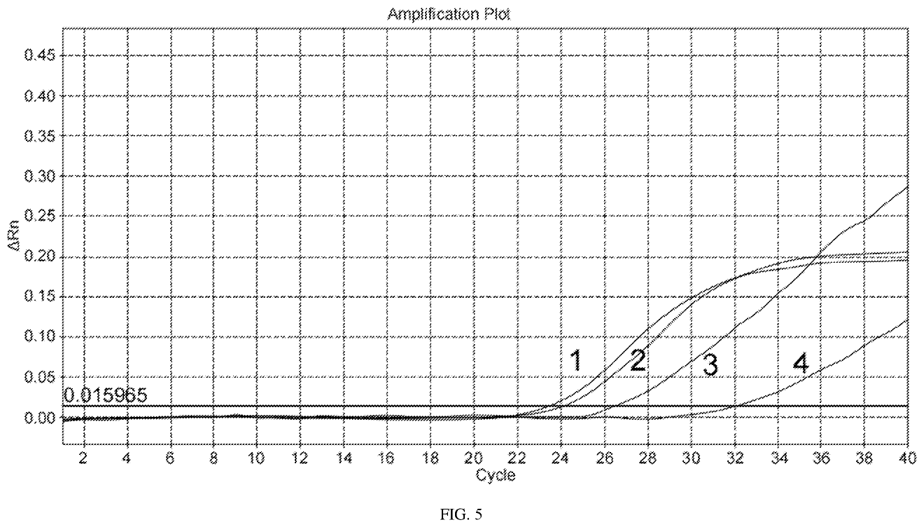

Compared with a domestic brand of histamine H4 receptor (HRH4) kit of enzyme-linked immunosorbent assay (ELISA), the results are shown in Table 5 and FIG. 5. The TaqMan real-time fluorescent quantitative RT-PCR detection method established in the present disclosure has better sensitivity and specificity than that of a counterpart reagent, and can effectively monitor the treatment effect.

TABLE 5
Comparison of clinical sample detection effect

| | | Product of the present | | Histamine H4 receptor | |
|---|---|---|---|---|---|
| | | disclosure | | (HRH4) kit of ELISA | |
| | | Results | positive/ | Result | positive/ |
| SN | Sample type | (copies/μL) | negative | (ng/mL) | negative |
| 1 | Case 1 before | 1154.669 | + | 678.3 | + |
| | treatment | | | | |
| 2 | Case 1 after | 960.673 | + | 153.2 | + |
| | treatment | | | | |
| 3 | Case 2 before | 911.955 | + | 487.3 | + |
| | treatment | | | | |
| 4 | Case 2 after | 496.380 | − | 37.6 | − |
| | treatment | | | | |
| 5 | Case 3 before | 1620.624 | + | 781.4 | + |
| | treatment | | | | |
| 6 | Case 3 after | 32.378 | − | 147.6 | + |
| | treatment | | | | |
| 7 | Case 4 | 1447.242 | + | 539.1 | + |
| 8 | Healthy control 1 | 756.543 | − | 356.4 | + |
| 9 | Healthy control 2 | 30.457 | − | 19.2 | − |
| 10 | Healthy control 3 | 61.125 | − | 28.7 | − |

### Comparative Example 1 Results of Amplification Using Other Non-Optimal Primers and Probes

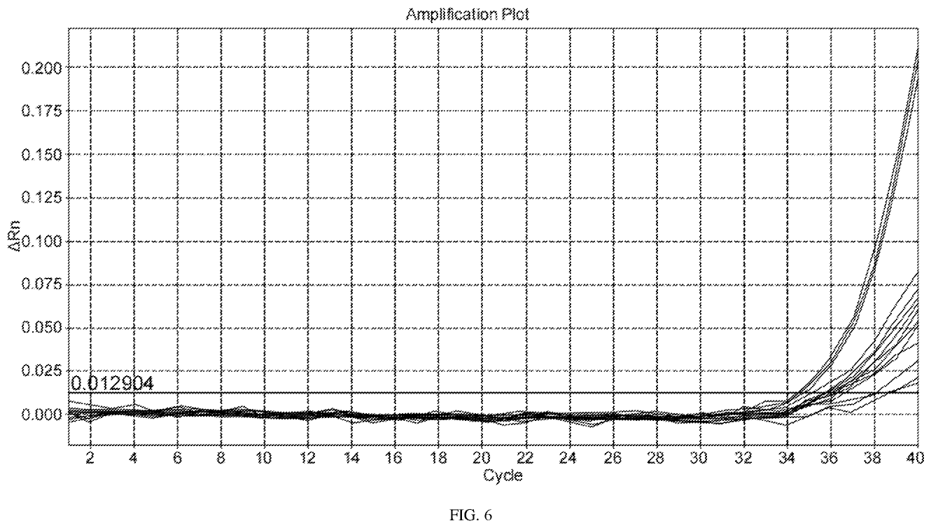

The primers and probes in the system used in the present disclosure in Example 5 were replaced with other non-optimal primers and probes. The results are shown in FIG. 6. When using non-optimal HRH4 primers and probes, the amplification results of the standard curve are poor, with almost no amplification.

Non-optimal HRH4 primers and probes were as follows:

HRH4-F (SEQ ID NO. 7):
GCCAGATACTAATAGCACAAT;
HRH4-R (SEQ ID NO. 8):
CCACAAAAGCTAAAATGACCAA;
and
H4-Probe (SEQ ID NO. 9):
(FAM)-AAGCACTCGTGTTACTTTAGCA-(BHQ1).

### Comparative Example 2 Comparison of the Effect of Enzyme Mixed Solution

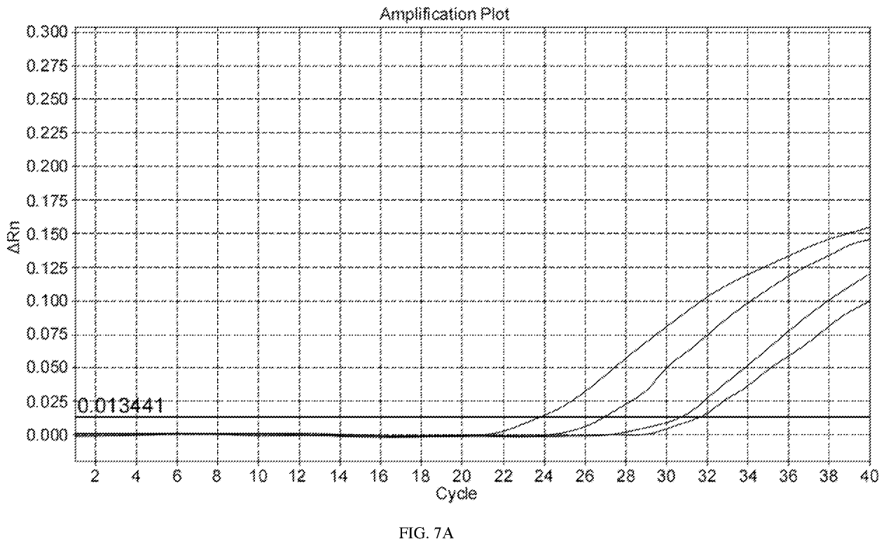

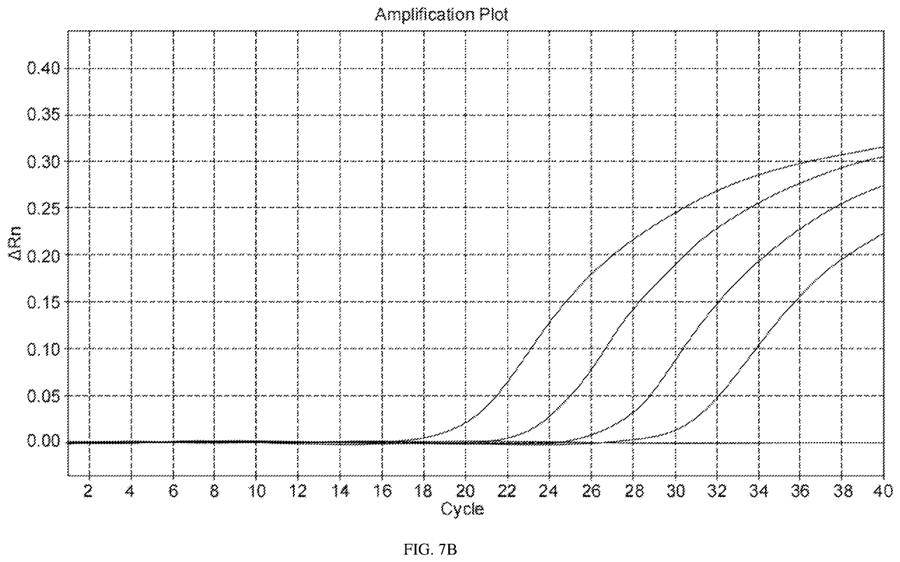

An amplification was conducted using a non-optimal ratio of enzyme mixed solution (the Taq enzyme, reverse transcriptase, RNase inhibitor and Taq enzyme antibody had a mass ratio of 16:3:5:1) and a best ratio of enzyme mixed solution on 4 gradients 1.0×10³-1.0×10⁶ copies/ill on a calibration curve. An amplification result using the non-optimal ratio of enzyme mixed solution is shown in FIG. 7A, and an amplification result using the best ratio of enzyme mixed solution is shown in FIG. 7B. It can be seen that the best enzyme mixed solution has a desirable amplification effect.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1.-14. (canceled)

15. A reagent for detecting an expression level of a human histamine receptor H4 (HRH4) messenger ribonucleic acid (mRNA), comprising a specific primer and a probe for a human histamine receptor HRH4, wherein the specific primer comprises an HRH4-F and an HRH4-R, and the probe comprises an H4-Probe; and
the HRH4-F has a nucleotide sequence shown in SEQ ID NO. 1, the HRH4-R has a nucleotide sequence shown in SEQ ID NO. 2 and the H4-Probe has a nucleotide sequence shown in SEQ ID NO. 3.

16. The reagent according to claim 15, further comprising a specific primer and a probe of a reference gene glyceraldehyde-3-phosphate dehydrogenase (GAPDH), wherein the specific primer of the reference gene GAPDH comprises a GAPDH-F and a GAPDH-R, and the probe of the reference gene GAPDH comprises a G-Probe; and
the GAPDH-F has a nucleotide sequence shown in SEQ ID NO. 4, the GAPDH-R has a nucleotide sequence shown in SEQ ID NO. 5 and the G-Probe has a nucleotide sequence shown in SEQ ID NO. ID NO. 6.

17. The reagent according to claim 15, wherein 5′-ends of the H4-Probe and G-Probe are separately labeled with different fluorescent reporter groups, and 3′-ends of the H4-Probe and G-Probe are labeled with a same quenching group or different quenching groups.

18. The reagent according to claim 16, wherein 5′-ends of the H4-Probe and G-Probe are separately labeled with different fluorescent reporter groups, and 3′-ends of the H4-Probe and G-Probe are labeled with a same quenching group or different quenching groups.

19. The reagent according to claim 17, wherein the fluorescent reporter group comprises a 6-carboxyfluorescein (FAM) or a 2,7-dimethyl-4,5-dichloro-6-carboxyfluorescein (JOE), and the quenching group comprises a Black Hole Quencher-1 (BHQ1).

20. The reagent according to claim 18, wherein the fluorescent reporter group comprises a 6-carboxyfluorescein (FAM) or a 2,7-dimethyl-4,5-dichloro-6-carboxyfluorescein (JOE), and the quenching group comprises a Black Hole Quencher-1 (BHQ1).

21. The reagent according to claim 15, wherein the HRH4-F, the HRH4-R, the H4-Probe, the GAPDH-F, the GAPDH-R and the G-Probe in the reagent have a concentration of 2.25 nM, 1.5 nM, 1.5 nM, 1 nM, 1 nM, and 1.5 nM, respectively.

22. The reagent according to claim 16, wherein the HRH4-F, the HRH4-R, the H4-Probe, the GAPDH-F, the GAPDH-R and the G-Probe in the reagent have a concentration of 2.25 nM, 1.5 nM, 1.5 nM, 1 nM, 1 nM, and 1.5 nM, respectively.

23. A kit for one-step detection of an expression level of a human histamine receptor H4 (HRH4) messenger ribonucleic acid (mRNA), comprising the reagent according to claim 15, a polymerase chain reaction (PCR) reaction solution, an enzyme mixed solution, a carboxy-X-rhodamine (ROX) reference dye and nuclease-free water.

24. The kit according to claim 23, further comprising a specific primer and a probe of a reference gene glyceraldehyde-3-phosphate dehydrogenase (GAPDH), wherein the specific primer of the reference gene GAPDH comprises a GAPDH-F and a GAPDH-R, and the probe of the reference gene GAPDH comprises a G-Probe; and
the GAPDH-F has a nucleotide sequence shown in SEQ ID NO. 4, the GAPDH-R has a nucleotide sequence shown in SEQ ID NO. 5 and the G-Probe has a nucleotide sequence shown in SEQ ID NO. ID NO. 6.

25. The kit according to claim 23, wherein 5′-ends of the H4-Probe and G-Probe are separately labeled with different fluorescent reporter groups, and 3′-ends of the H4-Probe and G-Probe are labeled with a same quenching group or different quenching groups.

26. The kit according to claim 25, wherein the fluorescent reporter group comprises a 6-carboxyfluorescein (FAM) or a 2,7-dimethyl-4,5-dichloro-6-carboxyfluorescein (JOE), and the quenching group comprises a Black Hole Quencher-1 (BHQ1).

27. The kit according to claim 23, wherein the HRH4-F, the HRH4-R, the H4-Probe, the GAPDH-F, the GAPDH-R and the G-Probe in the reagent have a concentration of 2.25 nM, 1.5 nM, 1.5 nM, 1 nM, 1 nM and 1.5 nM, respectively.

28. The kit according to claim 23, wherein the PCR reaction solution comprises a deoxy-ribonucleoside triphosphate (dNTP) mix, MgCl₂ and a buffer; and
the enzyme mixed solution comprises a Thermus aquaticus (Taq) enzyme, a reverse transcriptase, a ribonuclease (RNase) inhibitor and a Taq enzyme antibody with a mass ratio of 15:5:4:1.

29. The kit according to claim 23, further comprising an RNA standard of the human histamine receptor HRH4.

30. A method for detecting an expression level of a human histamine receptor H4 (HRH4) messenger ribonucleic acid (mRNA) in one step based on the reagent according to claim 15, comprising the following steps: preparing a same reaction system using a ribonucleic acid (RNA) extracted from a sample as a template, conducting a same quantitative real-time polymerase chain reaction (qRT-PCR), and determining the expression level of the human histamine receptor HRH4 mRNA using a standard curve constructed by a standard.

31. The method according to claim 30, wherein a process for constructing the standard curve comprises: preparing a reaction system using a RNA standard of the human histamine receptor HRH4 as a template, conducting a qRT-PCR, and constructing the standard curve using a logarithmic value of a copy number as an abscissa and using a Ct value as an ordinate.

32. The method according to claim 30, wherein the reaction system is 20 μl, comprising: 2.4 μl of the nuclease-free water, 10 μl of the PCR reaction solution, 0.5 μl of the enzyme mixed solution, 0.5 μl of the ROX reference dye, 2 μl of the reagent, and 5 μl of the template.

33. The method according to claim 30, wherein a qRT-PCR program comprises: 42° C. for 30 minutes; 95° C. for 1 minute; 95° C. for 5 seconds, 60° C. for 31 seconds, 40 cycles.

34. The method according to claim 30, wherein the standard curve is y=−3.177x+34.178, R²=0.996.
